(19) United States
(12) Patent Application Publication
(10) Pub. No.: US 2005/0148934 A1
(43) Pub. Date: Jul. 7, 2005

(54) Fluid infusion apparatus with an insulated patient line tubing for preventing heat loss

(75) Inventors: Paul William Martens, Pleasanton, CA (US); Joseph Coakley, Dublin, CA (US)

Correspondence Address: ROTHWELL, FIGG, ERNST & MANBECK, P.C., 1425 K STREET, N.W., SUITE 800, WASHINGTON, DC, 20005, US

(73) Assignee: Mallinckrodt Inc., St. Louis, MO (US)

(21) Appl. No.: 10/743,827

(22) Filed: Dec. 24, 2003

(51) Int. Cl. A61F 7/12

(52) U.S. Cl. 604/113

(57) ABSTRACT

A fluid warming and infusion system. According to one embodiment, the fluid warming and infusion system includes a container for storing a fluid to be infused into a patient, a fluid warmer for warming the fluid prior to the fluid being infused into the patient, a tubing for delivering the fluid to the patient after the fluid has been warmed by the fluid warmer, and a patient insertion device (e.g., a needle, or the like), which is connected to a distal end of the tubing, for insertion into the patient. After being warmed by the fluid warmer, the fluid flows through the tubing and is delivered into the patient by the insertion device. The tube includes a substantially thermally insulating component that prevents the warmed fluid from losing a substantial amount of heat as the fluid flows through the tube.

### BACKGROUND OF THE INVENTION

1. Field of the Invention

The present invention relates generally to the field of intravenous fluid delivery.

2. Description of the Background Art

Intravenous fluid delivery systems are systems used to infuse a fluid into the circulatory system of a patient. This may be done as part of medical treatment. The infusion may include infusion of fluids such as whole blood or blood components, saline solution, medications, or the like.

The warming of fluids that are infused into patients intravenously is a standard of care for operating room procedures where the flow rates are typically above about 13-15 mL/min. In the case of lower flow rates for adults, the amount of infused fluid when compared to the mass of the patient is generally deemed to be insignificant, and so warming of the fluid is not practiced. In the case of pediatric and neonatal patients, the comparison is different, and flow rate of less than 13-15 mL/min, down to as low as 1 or 2 mL/min are considered candidates for warming.

Conventionally, the infused fluid is warmed by a fluid warmer that is some distance away from the patient. After being heated by the fluid warmer, the fluid proceeds through a patient line and then into the patient. As the fluid proceeds through the patient line, the fluid loses heat by, for example, radiation and convection heat loss. This heat loss is problematic, particularly when the fluid flow rate is about 10 mL/min or less.

An investigation into geometric changes in the tubing of the patient line to prevent this heat loss has been made. As an example, reducing the diameter of the tubing ostensibly increases the velocity of the fluid, which means that the fluid spends less time in the tubing. Less time in the tubing should mean less heat loss by means of the radiation and convection mechanisms. However, there are limitations to this concept. For example, as the diameter of the tubing is decreased, the surface area to volume ratio gets geometrically larger, meaning that there is more surface area exposed for heat to be lost. Additionally, in very small diameters, there is a pressure build up due to the resistance of flow in a restricted cross sectional area.

Therefore, there remains a need in the art for an improved system for reducing heat loss in an intravenous fluid delivery system.

### SUMMARY OF THE INVENTION

An improved fluid warming and infusion system is provided by the present invention. According to one embodiment, the fluid warming and infusion system includes a container for storing a fluid to be infused into a patient, a fluid warmer for warming the fluid prior to the fluid being infused into the patient, a tubing for delivering the fluid to the patient after the fluid has been warmed by the fluid warmer, and a patient insertion device (e.g., a needle or the like), which is connected to a distal end of the tubing, for insertion into the patient, wherein, after being warmed by the fluid warmer, the fluid flows through the tubing and is delivered into the patient by the insertion device (the tubing itself is not inserted into the patient). Advantageously, the tubing includes a substantially thermally insulating component for use as a thermal insulator in preventing the fluid from losing a substantial amount of heat as the fluid flows through the tubing.

In another aspect, the invention provides a fluid administration set for use with a fluid warmer. According to one embodiment, the fluid administration set includes a heat exchanger cassette configured to be inserted into a fluid warmer and functioning to transfer heat to a fluid flowing there through, a fluid line having one end in fluid communication with an input port of the heat exchanger cassette and another end adapted for connection to a fluid source (e.g., a container storing a fluid), and a patient line having one end in fluid communication with an output port of the heat exchanger cassette and another end configured to mate with a patient insertion device. The patient line includes a tubing having a substantially thermally insulating component for use as a thermal insulator in preventing fluids flowing there through from losing a substantial amount of heat.

The above and other features and advantages of the present invention will be further understood from the following description of the preferred embodiments thereof, taken in conjunction with the accompanying drawings.

### BRIEF DESCRIPTION OF THE DRAWINGS

The accompanying drawings, which are incorporated herein and form part of the specification, illustrate various embodiments of the present invention and, together with the description, further serve to explain the principles of the invention and to enable a person skilled in the pertinent art to make and use the invention. In the drawings, like reference numbers indicate identical or functionally similar elements. Additionally, the left-most digit(s) of a reference number identifies the drawing in which the reference number first appears.

FIG. 1 is a schematic view of a fluid warming and delivery system of one embodiment of the present invention;

FIG. 2 is a schematic view of a fluid administration set of one embodiment of the present invention.

FIG. 3 is a longitudinal section view of one embodiment of an insulated tube that can be used in the construction of the fluid administration set.

FIG. 4 is a transverse section view of the insulated tube shown in FIG. 3.

FIG. 5 is a transverse section view of a second embodiment of the insulated tube shown in FIG. 3.

FIG. 6 is a transverse section view of a third embodiment of the insulated tube shown in FIG. 3.

FIG. 7 is a longitudinal section view of a fourth embodiment of an insulated tube that can be used in the construction of the fluid administration set.

FIG. 8 is a transverse section view of the insulated tubing shown in FIG. 7.

### DETAILED DESCRIPTION OF THE PREFERRED EMBODIMENTS

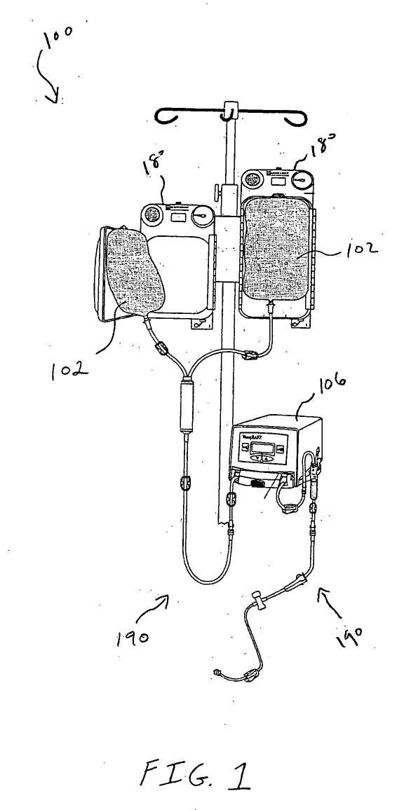

FIG. 1 shows a fluid warming and infusion system 100 according to one embodiment of the invention. As shown in FIG. 1, fluid warming and infusion system 100 includes a container 102 for holding a fluid, a fluid warmer 106 for transferring heat to the fluid, and fluid administration set 190 that is configured for use with fluid warmer 106. Container 102 is any suitable container for holding fluids, and, in one embodiment, fluid warmer 106 is the Warmflow® FW-588 fluid warmer available from Tyco Healthcare Group LP of Pleasanton, Calif. System 100 may include a pressure infusor 180 for forcing fluid to flow out of container 102 and into fluid administration set 190, however, in other embodiments, system 100 relies solely on gravity for this purpose.

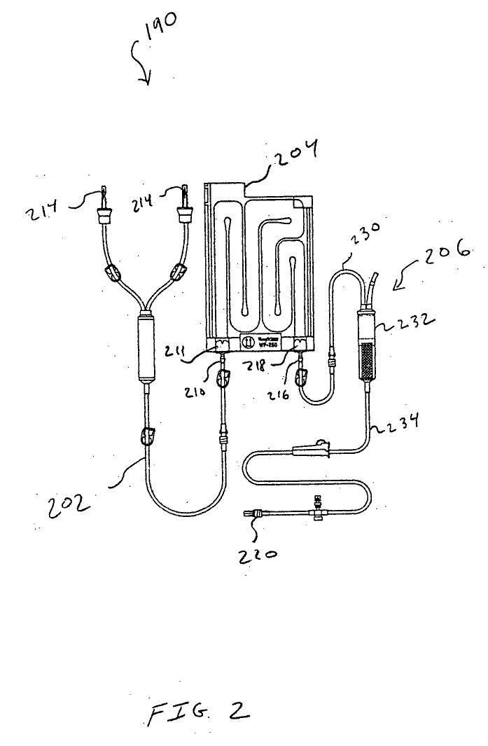

FIG. 2 is a schematic diagram further illustrating fluid administration set 190. As shown in FIG. 2, fluid administration set 190 includes a fluid line 202, a heat exchanger cassette 204, and a patient line 206. Heat exchanger cassette 204 is configured to be inserted into a fluid warmer, such as fluid warmer 106, and, after inserted into a fluid warmer, functions to transfer heat generated by heating elements in the fluid warmer to the fluid flowing through the cassette. Fluid line 202 has a first end 210 in fluid communication with an input port 211 of heat exchanger cassette 204 and a second end 214 adapted for connection to a fluid source (e.g., container 102). Patient line 206 has a first end 216 in fluid communication with an output port 218 of heat exchanger cassette 204 and a second end 220 configured to mate with a patient insertion device (not shown). In one embodiment, second end 220 of patient line 206 is a standard male (or female) luer connector.

In the embodiment shown in FIG. 2, patient line 206 includes a first tube 230, a drip chamber 232, which may provide a filtering function and/or an air elimination function, and a second tube 234, however, other configurations are contemplated, such as, for example, a single tube configuration without a chamber 232. Tube 230 provides a path for the fluid to flow from cassette 204 to chamber 232. More specifically, a proximal end of tube 230 forms end 216 of patient line 206 and a distal end of tube 230 is attached to an input port of chamber 232. Similarly, tube 234 provides a path for the fluid to flow out of chamber 232 and into the patient. More specifically, a proximal end of tube 234 is attached to an output port of chamber 232 and a distal end of tube 234 is attached to Luer connector 220.

Fluid administration set 190 may also include a vacuum source, such as a vacuum pump (not shown) to evacuate and maintain a vacuum in an insulating layer within tube 230 and/or 234. Another type of vacuum source, such as a syringe (not shown), could also be used instead of a vacuum pump.

In operation, the fluid in container 102 flows through fluid line 202 to heat exchanger cassette 204, which has been inserted into fluid warmer 106, through heat exchanger cassette 204 into patient line 206, and through patient line 206 to the patient. As the fluid passes through cassette 204, fluid warmer 106 warms the fluid to a predetermined temperature.

In preferred embodiments, tube 230 and/or 234 are insulated tubes that prevent fluids flowing there through from losing a substantial amount of heat.

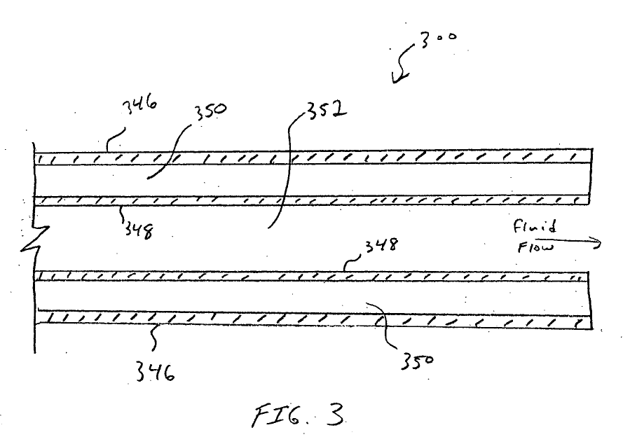

FIG. 3 is a longitudinal section view of one embodiment of an insulated tube 300 that can be used as tube 230 and/or 234. This embodiment of tube 300 has a coaxial construction, with an outer wall 346 and an inner wall 348. A fluid lumen 352 through which the warmed fluid may flow is formed by the inner wall 348. Fluid lumen 352 has an exit port adjacent the distal end of tube 300.

Advantageously, an annular insulating gap 350 is created between the outer wall 346 and the inner wall 348. The annular insulating gap 350 can be evacuated during manufacture of tube 300. If evacuated during manufacture, the annular insulating gap 350 could be sealed at its proximal and distal ends, thereby creating a constant, passive vacuum in the insulating gap 350.

Alternatively, the annular insulating gap 350 could be evacuated during use of tube 300, such as by the vacuum pump discussed above, or by a syringe. Conversely, the annular insulating gap 350 could be filled with an insulating material. Examples of suitable insulating materials include air, insulating foam such as polyurethane, or aerogel, and other insulating materials.

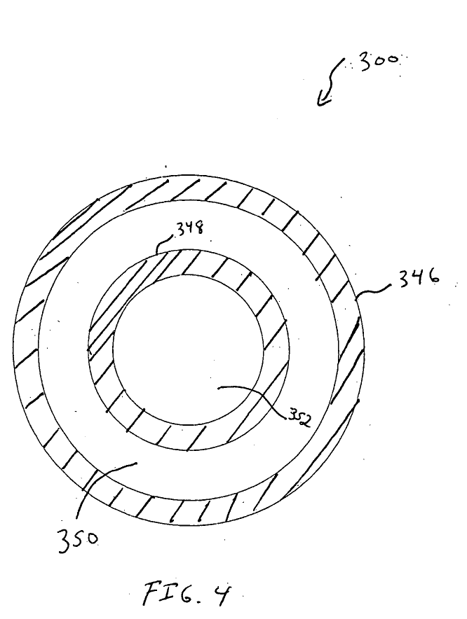

FIG. 4 is a transverse section of the embodiment of the insulated tube 300 shown in FIG. 3. FIG. 4 clearly shows the arrangement of the outer wall 346, the inner wall 348, the insulating annular gap 350, and the fluid lumen 352. As shown in FIG. 4, the circle formed by outer wall 346 may be concentric with the circle formed by inner wall 348.

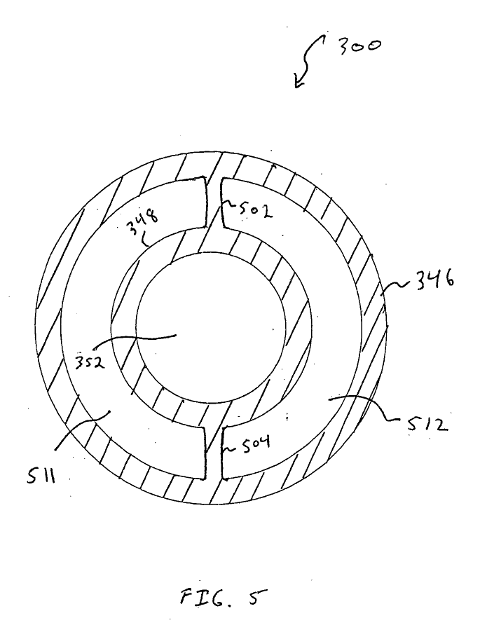

FIG. 5 is a transverse section of a second embodiment of the insulated tube 300. In this second embodiment, tube 300 further includes a first partition 502 and a second partition 504 connected between walls 346 and 348. Partitions 502 and 504 define a first insulating gap 511 and a second insulating gap 512 between walls 346 and 348. As shown in FIG. 5, partitions 502 and 504 may be spaced about 180 degrees apart. Partitions 502 and 504 may also function to maintain concentricity of the circles formed by walls 346/348.

Like insulating annular gap 350, insulating gaps 511 and 512 may be evacuated during manufacture of tube 300. If evacuated during manufacture, each of the insulating gaps could be sealed at their ends, creating a constant, passive vacuum in each of the insulating gaps. Alternatively, the insulating gaps 511, 512 could be evacuated during use of tube 300, such as by a vacuum pump or syringe. Conversely, the insulating gaps 511, 512 could be filled with an insulating material.

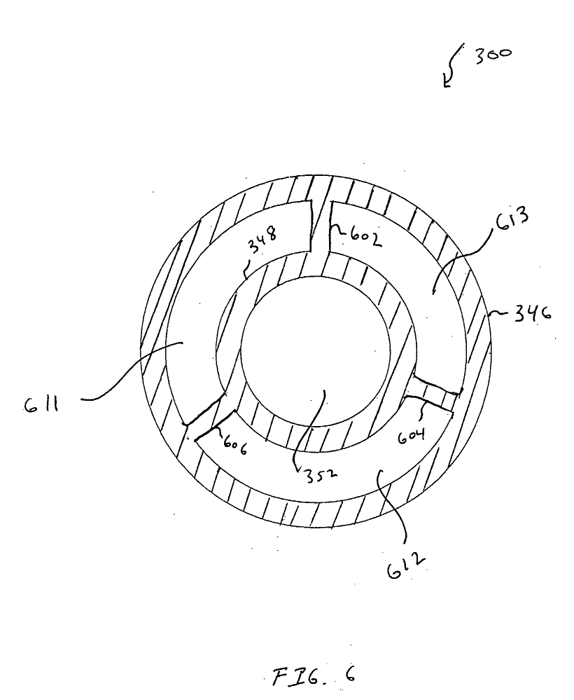

FIG. 6 is a transverse section of a third embodiment of the insulated tube 300. In this third embodiment, tube 300 further includes a first partition 602, a second partition 604, and a third partition 606 connected between walls 346 and 348. Partitions 602 and 604 define a first insulating gap 611, partitions 604 and 606 define a second insulating gap 612, and partitions 606 and 602 define a third insulating gap 613. Each of the insulating gaps partially surrounds and is parallel with fluid lumen 352. As shown in FIG. 6, partitions 602, 604 and 606 are spaced about 120 degrees apart from the nearest partition.

As with the embodiment shown in FIG. 5, insulating gaps 611-613 may be evacuated during manufacture of tube 300 or during its use. Conversely, the insulating gaps 611-613 could be filled with an insulating material.

It should be understood that tube 300 may include any number of partitions and, thereby, any number of insulating gaps between walls 348 and 346.

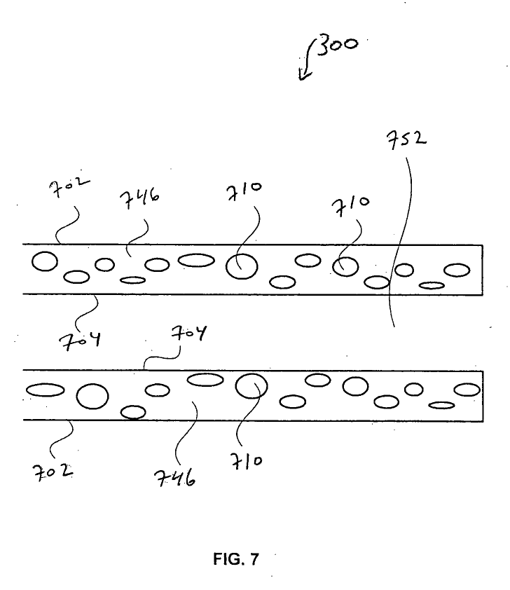

FIG. 7 is a longitudinal section view of a fourth embodiment of the insulated tube 300. In this embodiment, tube 300 includes only a single wall 746 having an outer surface 702 and an inner surface 704. A fluid lumen 752 through which the warmed fluid may flow is formed by the inner surface 704. Advantageously, during manufacture of tube 300 several insulating cavities 710 are formed within wall 746. The insulating cavities may be evacuated or filled with a gas, such as air.

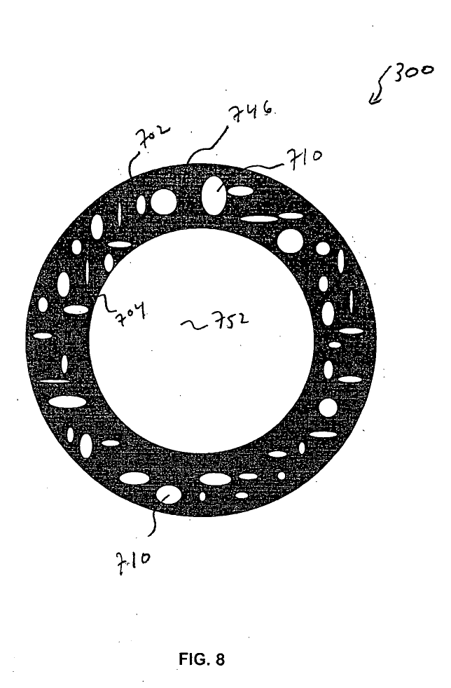

FIG. 8 is a transverse section of the embodiment of the insulated tube 300 shown in FIG. 7. FIG. 8 clearly shows the wall 746, the inner surface 704, the outer surface 702, and the insulating cavities 710.

While the invention has been described in detail above, the invention is not intended to be limited to the specific embodiments as described. It is evident that those skilled in the art may now make numerous uses and modifications of and departures from the specific embodiments described herein without departing from the inventive concepts.

1. A fluid warming and infusion system for infusing a warm fluid into a patient, comprising:
a device that transfers heat to the fluid, thereby warming the fluid; and
an insulated tube that transports the warmed fluid to the patient, wherein the insulated tube includes a substantially thermally insulating component that prevents the warmed fluid from losing a substantial amount of heat as the fluid flows through the tube.

2. The fluid warming and infusion system of claim 1, wherein the insulated tube has a configuration selected from the group consisting of:
A) an outer wall, an inner wall wherein an annular insulating gap is positioned between the outer wall and the inner wall, and a fluid lumen formed by the inner wall for transporting the warmed fluid.
B) an outer wall, an inner wall that forms a fluid lumen for transporting the warmed fluid, and at least two partitions connected between the inner and outer walls for forming at least two insulating gaps between the inner and outer walls; and
C) a single wall configuration wherein a plurality of insulating cavities exist within the wall.

3. The fluid warming and infusion system of claim 2, wherein each insulating gap or each of said cavities is evacuated and sealed so that a vacuum is created in the insulating gap(s) or cavities.

4. The fluid warming and infusion system of claim 2, wherein each insulating gap or each of said cavities is filled with an insulating material.

5. The fluid warming and infusion system of claim 4, wherein the insulating material is comprised primarily of air.

6. The fluid warming and infusion system of claim 4, wherein the insulating material comprises insulating foam.

7. The fluid warming and infusion system of claim 2, wherein there are only two partitions and the partitions are spaced about 180 degrees apart from each other.

9. The fluid warming and infusion system of claim 1, wherein said device is a heat exchanger cassette.

10. The fluid warming and infusion system of claim 1, wherein said device is a fluid warmer.

11. The fluid warming and infusion system of claim 1, further comprising a luer connector attached to a distal end of said insulated tube.

12. A method for infusing a warmed fluid into a patient utilizing the fluid warming and infusion system of claim 1, comprising a step of providing a patient line comprising a tube for delivering the warmed fluid to the patient, wherein the tube includes a substantially thermally insulating component that prevents the warmed fluid from losing a substantial amount of heat as the fluid flows through the tube.

13. The method of claim 12, wherein the insulated tube has a configuration selected from the group consisting of:
A) an outer wall, an inner wall wherein an annular insulating gap is positioned between the outer wall and the inner wall, and a fluid lumen formed by the inner wall for transporting the warmed fluid;
B) an outer wall, an inner wall that forms a fluid lumen for transporting the warmed fluid, and at least two partitions connected between the inner and outer walls for forming at least two insulating gaps between the inner and outer walls; and
C) a single wall configuration wherein a plurality of insulating cavities exist within the wall.

14. The method of claim 13, wherein each insulating gap or each of said cavities is evacuated and sealed so that a vacuum is created in the insulating gap(s) or cavities.

15. The method of claim 13, wherein each insulating gap or each of said cavities is filled with an insulating material.

16. The method of claim 15, wherein the insulating material is comprised primarily of air.

17. The method of claim 15, wherein the insulating material comprises insulating foam.

18. The method of claim 13, wherein there are only two partitions and the partitions are spaced about 180 degrees apart from each other.

19. A fluid administration set for use with the fluid warming and infusion system of claim 1, comprising:
a heat exchanger cassette having an input port and an output port;
a fluid line having a first end and a second end, the first end being configured to attach to an output port of a fluid container and the second end being in fluid communication with the input port of the cassette; and
a patient line having a first end and a second end, the first end being in fluid communication with the output port of the cassette, wherein
the patient line comprises a tube having a substantially thermally insulating component that prevents a warmed fluid from losing a substantial amount of heat as the fluid flows through the tube.

20. The fluid administration set of claim 19, wherein the insulated tube has a configuration selected from the group consisting of:
A) an outer wall, an inner wall wherein an annular insulating gap is positioned between the outer wall and the inner wall, and a fluid lumen formed by the inner wall for transporting the warmed fluid;
B) an outer wall, an inner wall that forms a fluid lumen for transporting the warmed fluid, and at least two partitions connected between the inner and outer walls for forming at least two insulating gaps-between the inner and outer walls; and
C) a single wall configuration wherein a plurality of insulating cavities exist within the wall.

21. The fluid administration set of claim 20, wherein each insulating gap or each of said cavities is evacuated and sealed so that a vacuum is created in the insulating gap(s) or cavities.

22. The fluid administration set of claim 20, wherein each insulating gap or each of said cavities is filled with an insulating material.

23. The fluid administration set of claim 22, wherein the insulating material is comprised primarily of air.

24. The fluid administration set of claim 22, wherein the insulating material comprises insulating foam.

25. The fluid administration set of claim 20, wherein there are only two partitions and the partitions are spaced about 180 degrees apart from each other.
